(19) United States
(12) Patent Application Publication
(10) Pub. No.: US 2022/0267846 A1
(43) Pub. Date: Aug. 25, 2022

(54) ANTI-POLLUTION CONSUMABLE AND METHOD FOR CLUSTERED REGULARLY INTERSPACED SHORT PALINDROMIC REPEATS (CRISPR) MOLECULAR DIAGNOSIS USING SAME

(71) Applicant: Xi'an Jiaotong University, Xi'an City (CN)

(72) Inventors: Niancai PENG, Xi'an City (CN); Fei HU, Xi'an City (CN); Yanfei LIU, Xi'an City (CN)

(21) Appl. No.: 17/566,566

(22) Filed: Dec. 30, 2021

(30) Foreign Application Priority Data
Feb. 22, 2021 (CN) 202110198318.5

(51) Int. Cl. C12Q 1/6876; C12N 9/22; C12N 15/11; B01L 3/00

(52) U.S. Cl. CPC C12Q 1/6876; C12N 9/22; C12N 15/11; B01L 3/5021; C12N 2310/20; C12N 2800/80; B01L 2300/043; B01L 2300/0832; B01L 2300/12; B01L 2200/141; B01L 2300/165

(57) ABSTRACT

The present disclosure provides an anti-pollution consumable and a method for Clustered Regularly Interspaced Short Palindromic Repeats (CRISPR) molecular diagnosis using the same, belonging to the technical field of nucleic acid detection and molecular diagnostics. The anti-pollution consumable includes an outer reaction tube, a sleeve and an inner reaction tube, where the inner reaction tube includes a second hollow cylindrical upper body and a second-type conical lower body sequentially from top to bottom; a top end of the second hollow cylindrical upper body is externally connected with a fixing ring perpendicular to the second hollow cylindrical upper body; a number of drain holes are provided at a bottom of the second-type conical lower body; the drain hole has a diameter of 0.01-0.8 mm; the drain hole is used for hydrophobic treatment; and the inner reaction tube is fixed inside the outer reaction tube through the sleeve.

### CROSS REFERENCE TO RELATED APPLICATION(S)

This patent application claims the benefit and priority of Chinese Patent Application No. 202110198318.5, filed on Feb. 22, 2021, the disclosure of which is incorporated by reference herein in its entirety as part of the present application.

### TECHNICAL FIELD

The present disclosure belongs to the technical field of nucleic acid detection and molecular diagnostics, in particular to an anti-pollution consumable and a method for Clustered Regularly Interspaced Short Palindromic Repeats (CRISPR) molecular diagnosis using the same.

### BACKGROUND ART

Fast, accurate, sensitive and quantitative detection of specific nucleic acid sequences is increasingly important in the fields such as diagnosis of human infectious diseases, food security, determination of pathogens, global biosecurity, tracking of biological pollutions in environmental analysis and environmental quality monitoring. The epidemic of Coronavirus Disease 2019 (COVID-19) has also brought a huge threat to people's lives. The rapid outbreak of the COVID-19 is caused by a lack of effective detection methods; at present, the standard detection method generally recognized by the world is polymerase chain reaction (PCR) detection. However, the PCR detection has a long detection cycle that takes about two to three hours to complete a nucleic acid test; in addition, the PCR detection is limited by the requirement of specialized technical personnel and large-scale equipment. In addition to traditional standard PCR detection, there are other mainstream nucleic acid amplification-based detection technologies such as a recombinase polymerase amplification (RPA) technology, a LAMP (loop-mediated isothermal amplification) technology, an RCA (rolling circle amplification) technology, a CPA (cross priming amplification) technology and a PSR (polymerase spiral reaction) technology. However, these methods also have certain advantages and disadvantages. For example, the RPA technology has fast response speed and exponential amplification, but has low sensitivity and cannot meet the requirements of clinical testing; for another example, the LAMP technology has relatively high sensitivity and specificity, but has a complicated primer design. Therefore, there is a lack of a simple and effective nucleic acid detection method that can shorten detection time, improve detection sensitivity and inhibit propagation rate of viruses.

Clustered Regularly Interspaced Short Palindromic Repeats (CRISPR) is an adaptive immune system widely found in archaea and bacteria. The CRISPR system was first discovered in the genome of E. coli in the early 1990s. In 2013, the targeted gene editing technology CRISPR/Cas9 was discovered by researchers and successfully applied. Since then, CRISPR technology has become the most popular gene editing tool. In 2017, Professor Zhang Feng, as the top international scholar in the field of gene editing, from the Broad Institute of Harvard University applied the CRISPR technology to the field of nucleic acid detection with his team. With the joint efforts of Professor Zhang Feng and Jennifer Doudna with her team (the University of California Berkeley), this technology has been used to detect nucleic acid molecules of various pathogens (such as Zika virus, Dengue virus and Mycobacterium tuberculosis). Since 2017, the two teams have published many high-level articles based on CRISPR nucleic acid detection technology in “Science”, “Nature” and their sub-journals. In 2018, an in vitro diagnostic technology for pathogens or tumors developed based on the CRISPR was selected as one of the “Top Ten Scientific and Technological Advances in the World in 2018” in a vote organized by academicians of the Chinese Academy of Sciences and the Chinese Academy of Engineering. Since then, various nucleic acid detection platforms based on CRISPR enzymes such as Cas12, Cas13 and Cas14 have also emerged.

Compared with traditional nucleic acid detection methods, the CRISPR technology shows great advantages in terms of detection cost, efficiency, portability, specificity and simplicity. Meanwhile, this technology has desirable biocompatibility to be combined with other technologies to make nucleic acid detection easier and more sensitive. This technology is also known as a next-generation novel nucleic acid detection technology. However, the CRISPR technology still has some defects. In order to improve the detection sensitivity, generally the nucleic acids need to be amplified before conducting the CRISPR nucleic acid detection, and the CRISPR reaction system is added to detect target nucleic acids after the amplification. That is, operations such as opening cap, transferring reagents and closing cap after the amplification are required, easily causing aerosol pollution in the laboratory. This not only leads to relatively high false positive in the follow-up results, but also easily causes cross-contamination of the laboratory and related equipment. In addition, it also takes a lot of time to complete a series of operations, resulting in waste of manpower and material resources. Therefore, it is urgent to find a convenient and feasible device or method to solve the aerosol pollution in the laboratory caused by opening cap, transferring nucleic acid samples and closing cap from the nucleic acid amplification to the nucleic acid detection in the CRISPR technology.

In 2017, Professor Zhang Feng, as the top international scholar in the field of gene editing, from the Broad Institute of Harvard University with his team disclosed [Gootenberg, J S et al. Nucleic acid detection with CRISPR-Cas13a/C2c2 [J]. Science 356, 438-442 (2017)], where an RPA reagent for nucleic acid amplification and a CRISPR reagent for nucleic acid detection were mixed, such that the nucleic acid amplification and the nucleic acid detection were conducted simultaneously in a same test tube. In this way, the nucleic acid amplification and the nucleic acid detection are combined into one step, avoiding the aerosol pollution caused by operations such as opening cap and transferring nucleic acid samples, and improving the efficiency of nucleic acid detection. However, this method reduces detection sensitivity. In 2019, Hui hui Liu, and Yong ming Wang et al. proposed a CRISPR/Cas12a-based method, called “Cas12aVDet”, referring to [Hui hui Liu, Yong ming Wang. et al. Cas12aVDet: A CRISPR/Cas12a-Based Platform for Rapid and Visual Nucleic Acid Detection [J]. American Chemical Society 91, 12156-12161(2019).]. This method is used for rapid nucleic acid detection, and can reduce experimental operations to avoid aerosol pollution caused by opening cap and the like and improve detection efficiency. In this method, an RPA reagent and a CRISPR reagent are integrated into one test tube in a single reaction system, but the CRISPR reagent is added to an inner wall of a uniquely-designed tube; after RPA reaction is completed, the CRISPR reagent is added to an RPA reaction solution by centrifugation to conduct nucleic acid detection. In this way, the detection can be completed within 30 min, while avoiding aerosol pollution caused by operations such as opening cap and transferring nucleic acid samples. However, this method is difficult to operate in actual use, and the CRISPR reagent may be mixed with the RPA reagent in advance to reduce sensitivity. Meng yao Zhang, and Cheng zhi Liu et al. developed a detection platform for detecting Vibrio parahaemolyticus by CRISPR and PCR technologies, referring to [Meng yao Zhang, Cheng zhi Liu. et al. Selective endpoint visualized detection of Vibrio parahaemolyticus with CRISPR/Cas12a assisted PCR using thermal cycler for on-site application[J]. Science Direct 214, 1873-3573 (2020).]. In this method, a CRISPR reagent is added to a test tube cap in advance, and a PCR reagent cap is closed to conduct PCR on a micro thermal cycler; through an adsorption force between droplets, the CRISPR reagent can be adsorbed on the test tube cap, thereby separating the two reagents. During the process, an upper cap of the thermal cycler is opened to avoid enzyme inactivation, such that the entire reaction is conducted partially on the thermal cycler and partially being exposed to the air. After the amplification is completed, the CRISPR reagent adsorbed on the test tube cap is thrown to a bottom of the test tube by centrifugation and mixed with the PCR reagent, and the nucleic acid detection is conducted by dUTP instead of dTTP in the PCR system, thereby avoiding the operations such as opening cap and transferring nucleic acid samples. However, being similar to adding the CRISPR reagent to the inner wall of the test tube, this method may also cause the CRISPR reagent and the RPA reagent to be mixed in advance and may have incorrect operations, resulting in reduced sensitivity.

Therefore, the existing technologies still cannot achieve pollution-free operations without causing false positive results and reducing sensitivity.

### SUMMARY

In view of this, the purpose of the present disclosure is to avoid laboratory aerosol pollution caused by operations such as opening cap, transferring nucleic acid samples and closing cap during nucleic acid detection based on the CRISPR technology. In the present disclosure, the provided anti-pollution consumable can make the detection system more robust, reduce experimental operations, prevent pollution and improve efficiency and accuracy, without reducing sensitivity and causing relatively high false positive results in nucleic acid detection based on the CRISPR technology.

To achieve the above objective, the present disclosure provides the following technical solution.

The present disclosure provides an anti-pollution consumable, including an outer reaction tube, a sleeve and an inner reaction tube, where

the outer reaction tube includes a tube cap, a first hollow cylindrical upper body, and a first-type conical lower body with a closed bottom sequentially from top to bottom;

the first-type conical lower body has a maximum inner diameter smaller than an inner diameter of the first hollow cylindrical upper body;

the sleeve has an outer diameter the same as an inner diameter of the first hollow cylindrical upper body;

the sleeve has a height less than that of the hollow cylindrical upper body;

the inner reaction tube includes a second hollow cylindrical upper body and a second-type conical lower body sequentially from top to bottom; a top end of the second hollow cylindrical upper body is externally connected with a fixing ring perpendicular to the second hollow cylindrical upper body; a number of drain holes are provided at a bottom of the second-type conical lower body; the drain hole has a diameter of 0.01-0.8 mm; and the drain hole is used for hydrophobic treatment;

the second hollow cylindrical upper body has an outer diameter less than or equal to an inner diameter of the sleeve; and the fixing ring has an outer diameter greater than the inner diameter of the sleeve and less than or equal to the inner diameter of the first hollow cylindrical upper body; and

the inner reaction tube is fixed inside the outer reaction tube through the sleeve.

Preferably, the hydrophobic treatment may include the following steps: 1) adding isopropanol to the inner reaction tube and centrifuging; and adding deionized water and centrifuging; 2) repeating the operations in step 1) for 2-3 times, and drying the inner reaction tube; and 3) adding a Teflon AF solution of a 0.5% FC-77 fluorinated oil (mass percentage) to a dried inner reaction tube and soaking.

Preferably, there may be 1-3 drain holes.

Preferably, the outer reaction tube may be made of polypropylene, and the sleeve and the inner reaction tube may be made of polymethyl methacrylate (PMMA).

Preferably, the inner reaction tube and the sleeve may be integrately synthesized.

Preferably, the inner reaction tube and the sleeve may be separately synthesized.

The present disclosure further provides a method for CRISPR molecular diagnosis using the anti-pollution consumable, including the following steps:

adding a CRISPR reagent to the outer reaction tube, and fixing the sleeve in the outer reaction tube;

adding a nucleic acid isothermal amplification reagent to the inner reaction tube, and fixing the inner reaction tube inside the sleeve through the fixing ring; and

adding a nucleic acid sample to be detected into the inner reaction tube, and covering the tube cap of the outer reaction tube; conducting nucleic acid isothermal amplification, centrifuging after the nucleic acid isothermal amplification is finished, and conducting nucleic acid detection.

Preferably, the centrifuging may be conducted at 600-6,000 rpm for 5-20 s.

Preferably, the nucleic acid isothermal amplification may be conducted by RPA isothermal amplification.

Preferably, the nucleic acid isothermal amplification may be conducted at 36-40° C. for 20-30 min.

Preferably, the nucleic acid detection may be conducted at 36-40° C. for 15-20 min.

The beneficial effects of the present disclosure are as follows: the anti-pollution consumable provided by the present disclosure includes an outer reaction tube, a sleeve and an inner reaction tube. The anti-pollution consumable is used for CRISPR molecular diagnosis, where operations such as opening cap, transferring nucleic acid samples and closing cap are reduced, and amplified samples and the CRISPR reagent can be mixed through simple centrifugation to simplify operation steps and improve efficiency of the nucleic acid detection; moreover, laboratory aerosol pollution caused by operations such as opening cap and transferring nucleic acid samples can be avoided to solve an important problem in the CRISPR nucleic acid detection technology.

### BRIEF DESCRIPTION OF THE DRAWINGS

FIG. 1A and FIG. 1B are schematic diagrams of an outer reaction tube of the present disclosure;

FIG. 2 is a stereoscopic diagram of the outer reaction tube of the present disclosure;

FIG. 3 is a schematic diagram of an inner reaction tube of the present disclosure;

FIG. 4 is a stereoscopic diagram of the inner reaction tube of the present disclosure;

FIG. 5 is a schematic diagram of a sleeve of the present disclosure;

FIG. 6 is a stereoscopic diagram of the sleeve and the inner reaction tube of the present disclosure;

FIG. 7 is a schematic diagram of a structure of the present disclosure;

FIG. 8 is a stereoscopic diagram of the present disclosure;

FIG. 9 is a schematic diagram of the outer reaction tube of the present disclosure added with a CRISPR reagent;

FIG. 10 is a schematic diagram of the sleeve combined with the outer reaction tube of the present disclosure;

FIG. 11 is a schematic diagram of the inner reaction tube of the present disclosure added with a nucleic acid amplification reagent;

FIG. 12 is a schematic diagram of the inner reaction tube combined with the sleeve and the outer reaction tube of the present disclosure;

FIG. 13 is a schematic diagram of the outer reaction tube of the present disclosure after a tube cap is covered;

FIG. 14 is a schematic diagram showing a nucleic acid sample after centrifugation entering the outer reaction tube to be mixed with the CRISPR reagent;

in FIG. 7 to FIG. 14, 1 refers to the tube cap, 2 refers to the outer reaction tube, 3 refers to the inner reaction tube, 4 refers to the sleeve, 5 refers to the drain hole, 6 refers to the CRISPR reagent, and 7 refers to the nucleic acid amplification reagent; and

FIG. 15 is an image of experimental results obtained using a mobile phone to take pictures of the present disclosure under blue light irradiation, where a left part is an image of a known positive target experiment, and a right part is an image of a blank control experiment with nuclease-free water.

### DETAILED DESCRIPTION OF THE EMBODIMENTS

The present disclosure provides an anti-pollution consumable, including an outer reaction tube, a sleeve and an inner reaction tube.

In the present disclosure, the anti-pollution consumable includes the outer reaction tube; the outer reaction tube includes a tube cap, a first hollow cylindrical upper body, and a first-type conical lower body with a closed bottom sequentially from top to bottom; the first-type conical lower body has a maximum inner diameter smaller than an inner diameter of the first hollow cylindrical upper body. The tube cap is fixedly connected to the outer reaction tube, and is used for sealing the outer reaction tube to ensure that the nucleic acid sample are not volatilized out of the tube to cause aerosol pollution. The outer reaction tube is preferably made of polypropylene and preferably manufactured by an injection molding process. The outer reaction tube is used for storing a CRISPR reagent and serves as a container for nucleic acid detection reaction after nucleic acid amplification is completed.

In the present disclosure, the anti-pollution consumable includes the sleeve, the sleeve has an outer diameter the same as an inner diameter of the first hollow cylindrical upper body of the outer reaction tube; and the sleeve has a height of less than that of the first hollow cylindrical upper body. The sleeve can be clamped on the first-type conical lower body inside the outer reaction tube, and can also clamp to fix and support the inner reaction tube to ensure that the inner reaction tube is half-suspended in the outer reaction tube and does not fall off. The outer reaction tube fixes supports the sleeve. The sleeve is preferably made of PMMA and preferably manufactured by an injection molding process.

In the present disclosure, the anti-pollution consumable includes the inner reaction tube. The inner reaction tube includes a second hollow cylindrical upper body and a second-type conical lower body sequentially from top to bottom; a top end of the second hollow cylindrical upper body is externally connected with a fixing ring perpendicular to the second hollow cylindrical upper body; the second hollow cylindrical upper body has an outer diameter less than or equal to an inner diameter of the sleeve; the fixing ring has an outer diameter greater than the inner diameter of the sleeve and less than or equal to the inner diameter of the first hollow cylindrical upper body; and the inner reaction tube is fixed inside the outer reaction tube through the sleeve. A number of drain holes are provided at a bottom of the second-type conical lower body; the drain hole has a diameter of 0.01-0.8 mm, preferably 0.4-0.6 mm, and most preferably 0.5 mm; and the drain hole is used for hydrophobic treatment. The hydrophobic treatment includes the following steps: 1) adding isopropanol to the inner reaction tube and centrifuging; and adding deionized water and centrifuging; 2) repeating the operations in step 1) for 2-3 times, and drying the inner reaction tube; and 3) adding a Teflon AF solution of a 0.5% FC-77 fluorinated oil (mass percentage) to a dried inner reaction tube and soaking.

In the present disclosure, the centrifuging is conducted at preferably 2,500-3,500 rpm, more preferably 3,000 rpm for preferably 8-12 sec, more preferably 10 sec; the centrifuging is to drain the isopropanol or the deionized water through the drain holes; the isopropanol is a cleaner and an oil remover, and the deionized water is to clean residual isopropanol; the FC-77 fluorinated oil is preferably purchased from Minnesota Mining and Manufacturing Company (3M Company, the United States), and is to conduct hydrophobic treatment on the drain holes. In step 2), the drying is conducted preferably by nitrogen; the soaking in the Teflon AF solution of a 0.5% FC-77 fluorinated oil (mass percentage) is conducted preferably for 0.5-1.5 h, more preferably 1 h; after the soaking, preferably the inner reaction tube is dried by nitrogen, and autoclaved for later use.

In the present disclosure, the drain hole has a small pore size. Without the interference of strong external forces, due to the surface tension of droplets and the atmospheric pressure, the drain hole can prevent the nucleic acid amplification reagent from flowing from the inner reaction tube to the outer reaction tube. The inner reaction tube is preferably made of PMMA and preferably manufactured by an injection molding process. The inner reaction tube is used for storing the nucleic acid amplification reagent, and also serves as a reaction container for the nucleic acid amplification.

In the present disclosure, optionally, the inner reaction tube and the sleeve are integrately synthesized and fixedly connected, or separately synthesized and detachably connected.

The present disclosure further provides a method for CRISPR molecular diagnosis using the anti-pollution consumable, including the following steps: adding a CRISPR reagent to the outer reaction tube, and fixing the sleeve in the outer reaction tube; adding a nucleic acid amplification reagent to the inner reaction tube, and fixing the inner reaction tube inside the sleeve through the fixing ring; and adding a nucleic acid sample to be detected into the inner reaction tube, and covering the tube cap of the outer reaction tube; conducting nucleic acid amplification, centrifuging after the nucleic acid amplification is finished, and conducting nucleic acid detection.

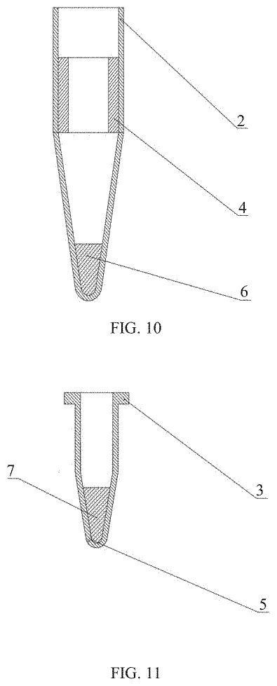

In the present disclosure, the CRISPR reagent is added to the outer reaction tube, and the sleeve is fixed in the outer reaction tube. There is no special limitation on specific composition and concentration of the CRISPR reagent, and conventional CRISPR reagents in the art can be used; the CRISPR reagent has a volume of preferably 20 μl; the CRISPR reagent is preferably added to the bottom of the outer reaction tube; the schematic diagram of the outer reaction tube added with the CRISPR reagent is shown in FIG. 9; the schematic diagram of the sleeve combined with the outer reaction tube is shown in FIG. 10.

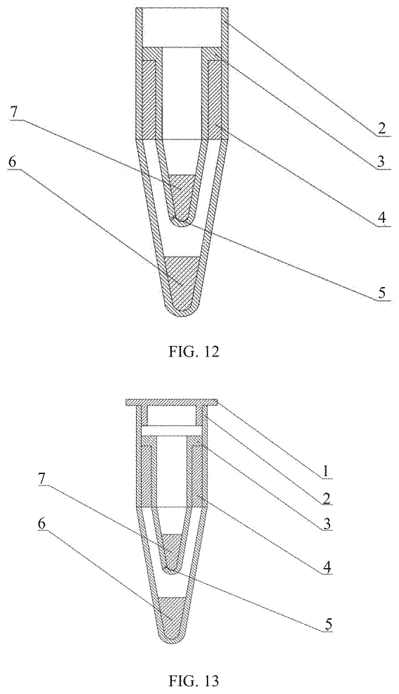

In the present disclosure, the nucleic acid amplification reagent is added to the inner reaction tube, and the inner reaction tube is fixed inside the sleeve through the fixing ring. There is no special limitation on specific composition and concentration of the nucleic acid amplification reagent, and conventional nucleic acid amplification reagents in the art can be used. The nucleic acid amplification reagent has a volume of preferably 10 μl. The nucleic acid amplification reagent is preferably added to the bottom of the inner reaction tube. The schematic diagram of the inner reaction tube added with the nucleic acid amplification reagent is shown in FIG. 11; the schematic diagram of the inner reaction tube combined with the sleeve and the outer reaction tube is shown in FIG. 12.

In the present disclosure, the nucleic acid sample to be detected is added into the inner reaction tube, and the tube cap of the outer reaction tube is covered. There is no special limitation on the nucleic acid sample to be detected, and any kind of nucleic acid sample to be detected can be used. The nucleic acid sample to be detected has a volume of preferably 2 μl. The schematic diagram of the outer reaction tube after the tube cap is covered is shown in FIG. 13.

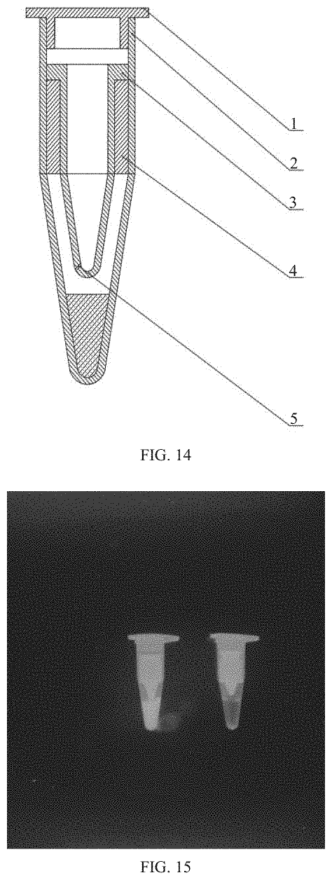

In the present disclosure, the nucleic acid amplification is conducted after the tube cap of outer reaction tube is covered, centrifuging is conducted after the nucleic acid amplification is finished, and nucleic acid detection is conducted. The nucleic acid amplification is conducted at preferably 36-40° C. for 20-30 min, more preferably 39° C. for 20 min; the centrifuging is conducted at preferably 600-6,000 rpm, more preferably 1,500 rpm for preferably 5-20 sec, more preferably 10 sec. The schematic diagram showing the nucleic acid sample after centrifugation entering the outer reaction tube to be mixed with the CRISPR reagent is shown in FIG. 14. The nucleic acid detection is conducted at preferably 37° C. for 20 min. After the nucleic acid detection is finished, results can be observed.

Through the anti-pollution consumable and the method provided by the present disclosure, a seamless connection can be realized from nucleic acid sample amplification to nucleic acid sample detection, and manual operations are reduced to save the time and improve the efficiency of nucleic acid detection. Meanwhile, the anti-pollution consumable and the method can avoid the aerosol pollution in the laboratory caused by opening cap, transferring nucleic acid samples and closing cap from the nucleic acid amplification to the nucleic acid detection.

The technical solution provided by the present disclosure will be described in detail below with reference to examples, but the examples should not be construed as limiting the protection scope of the present disclosure.

### Example 1

A size and a preparation method of a specific structure of each part of an anti-pollution consumable were provided.

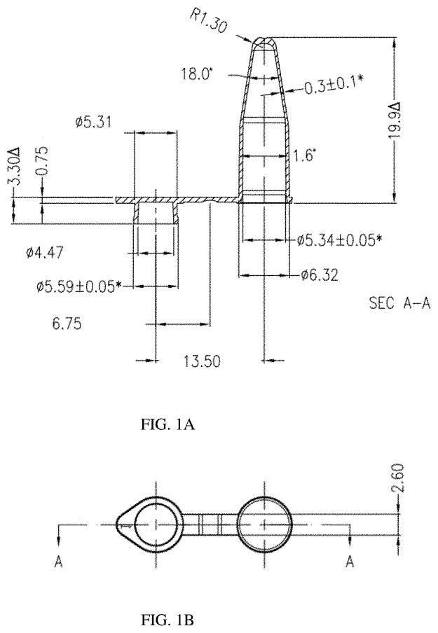

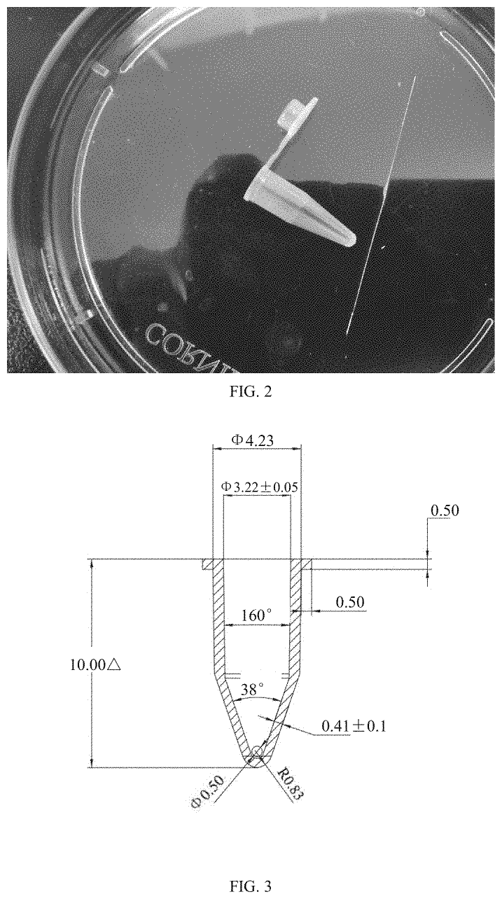

1. An outer reaction tube was made of high-quality polypropylene material; the high-quality polypropylene material was put into an injection molding machine for heating and melting, a product was extruded into a mold cavity by a screw under pressure, and processed through cooling and molding; this part was suitable for conventional PCR, had a specific size shown in FIG. 1A and FIG. 1B(mm), and had a stereoscopic diagram shown in FIG. 2.

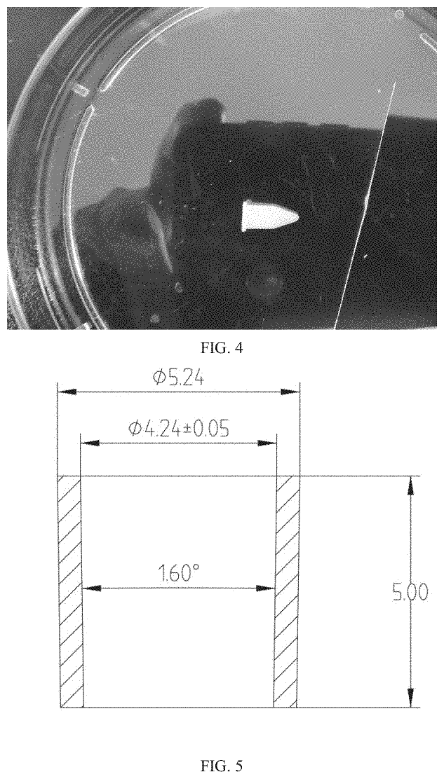

2. An inner reaction tube was made of high-quality PMMA material; the high-quality PMMA material was put into an injection molding machine for heating and melting, a product was extruded into a mold cavity by a screw under pressure, and processed through cooling and molding; the inner reaction tube was provided with drain holes on one side, had a specific size shown in FIG. 3, and had a stereoscopic diagram shown in FIG. 4.

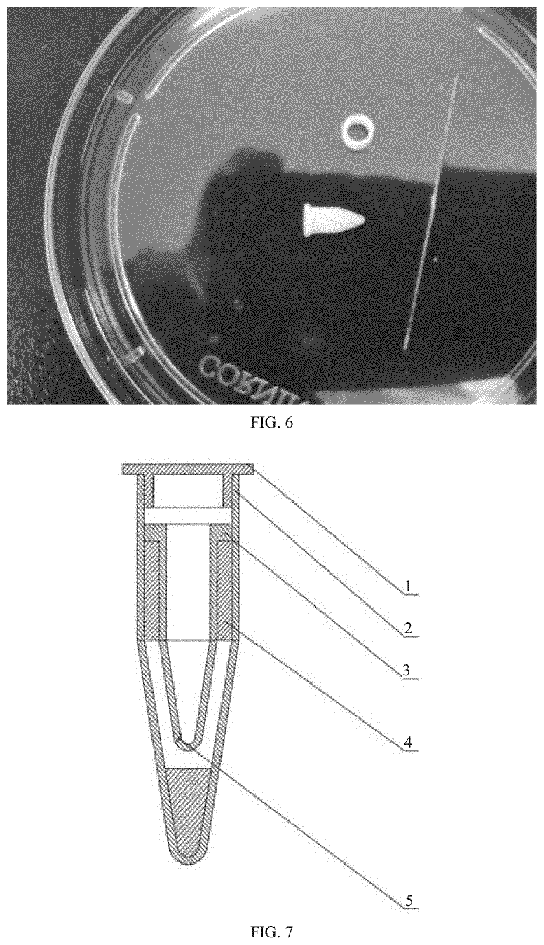

3. A sleeve was made of high-quality PMMA material; the high-quality PMMA material was put into an injection molding machine for heating and melting, a product was extruded into a mold cavity by a screw under pressure, and processed through cooling and molding; the sleeve had a specific size shown in FIG. 5, and the sleeve and the inner reaction tube had a stereoscopic diagram shown in FIG. 6.

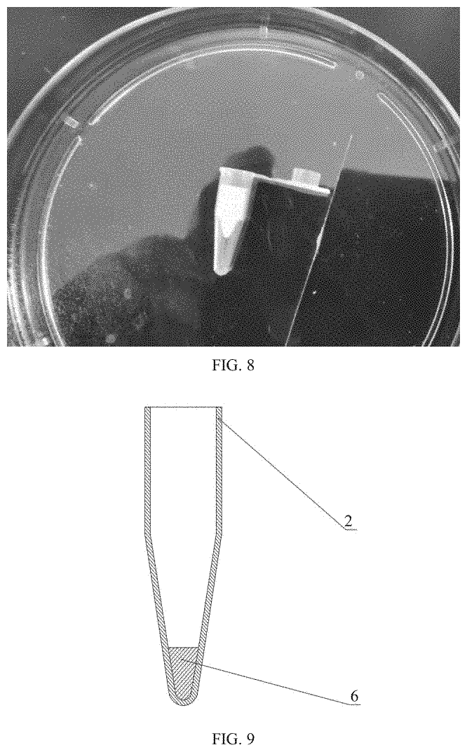

4. The outer reaction tube, the sleeve, and the inner reaction tube were nested into each other, and a stereoscopic diagram after the nesting was shown in FIG. 8.

### Example 2

A structure of the anti-pollution consumable and a schematic diagram of each step were shown in FIG. 9 to FIG. 14. The operation steps were as follows: a configured CRISPR reagent 6 was added to an outer reaction tube 2, and a sleeve 4 was added to the outer reaction tube 2. The sleeve 4 could be stuck inside the outer reaction tube 2 to be fixed. A configured RPA nucleic acid amplification reagent 7 without nucleic acid sample was added to an inner reaction tube 3, where the RPA nucleic acid amplification reagent 7 was added to a bottom of the inner reaction tube 3 as far as possible; due to the surface tension of droplets and the atmospheric pressure, although there is a drain hole at the bottom of the inner reaction tube 3, the RPA nucleic acid amplification reagent 7 was not flowing out along the drain hole 5. The inner reaction tube 3 was put into the outer reaction tube 2 for fixing. The nucleic acid sample to be amplified was added to the inner reaction tube 3, where the nucleic acid sample to be amplified was added to the bottom of the inner reaction tube 3 as far as possible; a tube cap was covered, and the outer reaction tube 2 was gently shook from side to side to mix the RPA nucleic acid amplification reagents evenly. The outer reaction tube 2 was put into a constant temperature centrifuge device at 39° C. and incubated for 20 min; after the incubation was completed, a centrifuge was started to centrifuge the entire outer reaction tube at 1,500 rpm for 10 sec; under the action of centrifugal force, a nucleic acid sample after amplification flew to an interior of the outer reaction tube 2 through the drain hole of the inner reaction tube 3, and mixed with the CRISPR reagent. Incubation was conducted for 20 min at a constant temperature of 37° C. to observe experimental results. Under blue light irradiation, taking pictures was conducted using a mobile phone, and test results were shown in FIG. 15. It can be clearly seen that the sample containing target molecules has obvious fluorescence after the reaction, while the control group not containing the target molecules has no fluorescence.

Now taking hepatitis B virus (HBV) as an example, a workflow of the anti-pollution consumable for CRISPR was described.

1. As shown in FIG. 9, a CRISPR reaction solution 6 was configured in a certain ratio and sequence at the bottom of the outer reaction tube 2.

2. As shown in FIG. 10, the sleeve 4 was put into the outer reaction tube 2 to fix the sleeve 4.

3. As shown in FIG. 11, the RPA nucleic acid amplification reagent 7 was configured in a certain ratio and sequence at the bottom of the inner reaction tube 3, but the nucleic acid sample was not added during this process; and the reagent was repeatedly blown twice to mix the reagent evenly.

4. As shown in FIG. 12, the inner reaction tube 3 was put into the outer reaction tube 2; the inner reaction tube 3 was caught by the sleeve 4 and suspended inside the outer reaction tube 2, and the inner reaction tube 2 was fixed.

5. The nucleic acid sample to be amplified was added to the bottom of the inner reaction tube 2, and the reagent was blown repeatedly twice to mix the reagent evenly.

6. As shown in FIG. 13, the outer reaction tube cap was covered, the entire PCR test tube was put into a 39° C. constant temperature centrifuge to be incubated for 20 min, to amplify the nucleic acid sample.

7. As shown in FIG. 14, the centrifuge is turned on, and under the action of centrifugal force, the amplified nucleic acid sample entered the outer reaction tube through the drain hole, and was evenly shook, such that the nucleic acid sample and the PCR reagent were homogeneously mixed.

8. The entire PCR test tube was incubated at 37° C. for 20 min, and the fluorescence could be observed to conveniently and quickly detect the nucleic acid.

For the following 20 unknown samples, parallel detection was conducted using a real-time fluorescence quantitative PCR technology and the technical method of the present disclosure, respectively.

For an unknown sample, the fluorescence quantitative PCR detection included the specific steps as follows.

1) Corresponding real-time fluorescence quantitative PCR reagents were prepared, including a Taq enzyme, a PCR buffer, dNTP, an HBV upstream primer, an HBV downstream primer, a 20×_{SYBRGreen} dye, double distilled water and a target.

2) Two PCR test tubes were labeled separately, namely a outer reaction tube 1 and a outer reaction tube 2.

3) A corresponding amplification reagent was configured and added by the following sequence into the 0.2 ml outer reaction tube 1.

| No. | Reagent name | Volume (μl) |
|---|---|---|
| 1 | 10X PCR buffer | 15 |
| 2 | dNTP (2.5 Mm) | 12 |
| 3 | Taq enzyme (5 u/μl) | 3 |
| 4 | 20X_{SYBRGreen} dye | 3 |
| 5 | HBV upstream primer (10 μM) | 7.5 |
| 6 | HBV downstream primer (10 μM) | 7.5 |
| 7 | Double distilled water | 72 |
| 8 | Total | 120 |

4) The outer reaction tube 1 (containing reagents) was placed in a shaker, shook well for 10 sec, and centrifuged for 12 sec in a small centrifuge at 2,000 rpm/min.

5) 60 μl of a centrifuged reagent from the outer reaction tube 1 was added to the outer reaction tube 2, 15 μl of double distilled water was added to the outer reaction tube 1, 15 μl of the corresponding target sample was added to the outer reaction tube 2, and the tube cap was covered.

6) The outer reaction tube 1 containing reagents and the outer reaction tube 2 containing reagents were placed in a shaker, shook well for 10 sec, and centrifuged for 12 sec in a small centrifuge at 2,000 rpm/min.

7) Six tubes were marked as a, b, c, d, e and f.

8) 20 μl of the reagent in the outer reaction tube 1 was added to tubes a, b and c, and 20 μl of the reagent in the outer reaction tube 2 was added to tubes d, e and f.

9) The tubes a, b, c, d, e and f were put into the centrifuge and centrifuged for 15 sec at 2,000 rpm/min.

10) The tubes a, b, c, d, e and f were put into a real-time fluorescence PCR machine for detection, and a program was set as follows:

95° C. for 5 min;

94° C. for 6 sec;

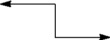

60° C. for 30 sec, detecting fluorescence; conducting 45 cycles

11) The results of the experiment were observed to determine whether it is positive or negative.

Moreover, detection verification was conducted using the method described in the present disclosure. The results of the above PCR detection method are shown in Table 1, where + represents that the result is positive, and − represents that the result is negative.

TABLE 1
Detection results of nucleic acid

| | PCR nucleic acid | CRISPR technology-based |
|---|---|---|
| No. | detection +/− | nucleic acid detection +/− |
| 1 | + | + |
| 2 | + | + |
| 3 | − | − |
| 4 | − | − |
| 5 | + | + |
| 6 | − | − |
| 7 | + | + |
| 8 | − | − |
| 9 | + | + |
| 10 | + | + |
| 11 | + | + |
| 12 | + | + |
| 13 | − | − |
| 14 | + | + |
| 15 | + | + |
| 16 | + | + |
| 17 | + | + |
| 18 | − | − |
| 19 | + | + |
| 20 | + | + |

After comparing with results measured by a standard PCR technology, this method has the results consistent with those of the standard PCR-based nucleic acid detection. It is proved that this method can achieve nucleic acid detection with accurate experimental results. At the same time, the cap is covered during the entire process from the end of the amplification to the realization of CRISPR detection, the laboratory aerosol pollution caused by the leakage of amplicons is eliminated, such that the results are highly reliable. In addition, the reliability of the method in preventing laboratory aerosol pollution was studied as follows, and the experimental results obtained also prove that the method has desirable reliability in preventing the aerosol pollution.

In order to verify the reliability of the method in preventing aerosol pollution, in a confined space, corresponding detection of the same nucleic acid sample was conducted under blue light irradiation using the method multiple times; and in each detection, a negative control experiment was set up, where the negative control used nuclease-free water instead of the nucleic acid sample, and other conditions remained the same; after multiple experiments (detection times >10), the results showed that the negative control of this method did not show false positive results. Meanwhile, the previous CRISPR detection method was compared with this method; being different with this method, the previous CRISPR detection technology had operations of cap opening and sample adding, which might increase the possibility of laboratory aerosol pollution to a certain extent. After the experiment, it was found that in the second or third nucleic acid sample detection, the negative control experiment with nuclease-free water had the false positive results. The comparison of the two experimental results proves that the method is highly reliable in preventing the laboratory aerosol pollution. The results are shown in Table 2 and Table 3, where + represents that the results are positive, and − represents that the results are negative.

TABLE 2
Negative and positive detection results of the anti-pollution
consumable and method provided by the present disclosure

| Number of | Positive target | Nuclease-free water |
|---|---|---|
| experiment | test results | test results |
| 1 | + | − |
| 2 | + | − |
| 3 | + | − |
| 4 | + | − |
| 5 | + | − |
| 6 | + | − |
| 7 | + | − |
| 8 | + | − |
| 9 | + | − |
| 10 | + | − |
| 11 | + | − |

TABLE 3
Negative and positive detection results of the consumable and
method of the conventional CRISPR nucleic acid detection

| Number of | Positive target | Nuclease-free water |
|---|---|---|
| experiment | test results | test results |
| 1 | + | − |
| 2 | + | − |
| 3 | + | + |
| 4 | + | + |
| 5 | + | + |
| 6 | + | + |
| 7 | + | + |
| 8 | + | + |
| 9 | + | + |
| 10 | + | + |
| 11 | + | + |

Moreover, the detection sensitivity of nucleic acid samples was explored using this method. An initial nucleic acid sample was configured at a concentration of 2×10⁵ copies per microliter, and detection was conducted using this method under blue light irradiation, and the result showed a clear positive fluorescent signal; the initial nucleic acid sample was diluted tenfold, and the nucleic acid sample detection was conducted sequentially according to the concentrations from high to low starting from 2×10⁴ copies per microliter, and the experimental results were shown in Table 4. The results show that when the nucleic acid concentration is 2×10⁰ copies per microliter, obvious experimental results can be observed; but when the nucleic acid concentration is 2×10⁻¹ copies per microliter, no experimental result can be observed. Therefore, this method has a sensitivity of at least 2×10⁰ copies per microliter.

TABLE 4
Determination results of sample concentrations

| | Nucleic acid sample concentration | |
|---|---|---|
| No. | (copies per microliter) | Detection results |
| 1 | 2 × 10⁵ | + |
| 2 | 2 × 10⁴ | + |
| 3 | 2 × 10³ | + |
| 4 | 2 × 10² | + |
| 5 | 2 × 10¹ | + |
| 6 | 2 × 10⁰ | + |
| 7 | 2 × 10⁻¹ | − |

The foregoing are merely descriptions of preferred embodiments of the present disclosure. It should be noted that several improvements and modifications can be made by a person of ordinary skill in the art without departing from the principle of the present disclosure, and these improvements and modifications shall also be deemed as falling within the protection scope of the present disclosure.

1. An anti-pollution consumable comprising:
an outer reaction tube including a tube cap, a first hollow cylindrical upper body, and a first-type conical lower body with a closed bottom sequentially from top to bottom, the first-type conical lower body having a maximum inner diameter smaller than an inner diameter of the first hollow cylindrical upper body;
a sleeve having an outer diameter the same as the inner diameter of the first hollow cylindrical upper body of the outer reaction tube, the sleeve having a height less than a height of the first hollow cylindrical upper body; and
an inner reaction tube wherein having a second hollow cylindrical upper body and a second-type conical lower body sequentially from top to bottom, a top end of the second hollow cylindrical upper body being externally connected with a fixing ring perpendicular to the second hollow cylindrical upper body, one or more drain holes being provided at a bottom of the second-type conical lower body, each drain hole having a diameter of 0.01-0.8 mm, and each drain hole being used for hydrophobic treatment,
the second hollow cylindrical upper body having an outer diameter less than or equal to an inner diameter of the sleeve,
the fixing ring having an outer diameter greater than the inner diameter of the sleeve and less than or equal to the inner diameter of the first hollow cylindrical upper body, and
the inner reaction tube being fixed inside the outer reaction tube through the sleeve.

2. The anti-pollution consumable according to claim 1, wherein the hydrophobic treatment comprises the following steps: 1) adding isopropanol to the inner reaction tube and centrifuging; and adding deionized water and centrifuging; 2) repeating the operations in step 1) for 2-3 times, and drying the inner reaction tube; and 3) adding a Teflon AF solution of a 0.5% FC-77 fluorinated oil (mass percentage) to a dried inner reaction tube and soaking.

3. The anti-pollution consumable according to claim 1, wherein there are one to three drain holes.

4. The anti-pollution consumable according to claim 1, wherein the outer reaction tube is made of polypropylene, and the sleeve and the inner reaction tube are made of polymethyl methacrylate (PMMA).

5. The anti-pollution consumable according to claim 1, wherein the inner reaction tube and the sleeve are integratedly synthesized.

6. The anti-pollution consumable according to claim 1, wherein the inner reaction tube and the sleeve are separately synthesized.

7. A method for Clustered Regularly Interspaced Short Palindromic Repeats (CRISPR) molecular diagnosis, the method comprising the following steps:
providing an anti-pollution consumable comprising:
an outer reaction tube including a tube cap, a first hollow cylindrical upper body, and a first-type conical lower body with a closed bottom sequentially from top to bottom, the first-type conical lower body having a maximum inner diameter smaller than an inner diameter of the first hollow cylindrical upper body;
a sleeve having an outer diameter the same as the inner diameter of the first hollow cylindrical upper body of the outer reaction tube, the sleeve having a height less than a height of the first hollow cylindrical upper body; and
an inner reaction tube, wherein having a second hollow cylindrical upper body and a second-type conical lower body sequentially from top to bottom, a top end of the second hollow cylindrical upper body being externally connected with a fixing ring perpendicular to the second hollow cylindrical upper body, one or more drain holes being provided at a bottom of the second-type conical lower body, each drain hole having a diameter of 0.01-0.8 mm, and each drain hole being used for hydrophobic treatment,
the second hollow cylindrical upper body having an outer diameter less than or equal to an inner diameter of the sleeve,
the fixing ring having an outer diameter greater than the inner diameter of the sleeve and less than or equal to the inner diameter of the first hollow cylindrical upper body, and
the inner reaction tube being fixed inside the outer reaction tube through the sleeve;
adding a CRISPR reagent to the outer reaction tube, and fixing the sleeve in the outer reaction tube;
adding a nucleic acid isothermal amplification reagent to the inner reaction tube, and fixing the inner reaction tube inside the sleeve through the fixing ring;
adding a nucleic acid sample to be detected into the inner reaction tube, and covering the tube cap of the outer reaction tube;
conducting nucleic acid isothermal amplification;
centrifuging after the nucleic acid isothermal amplification is finished; and
conducting nucleic acid detection.

8. The method according to claim 7, wherein the centrifuging is conducted at 600-6,000 rpm for 5-20 seconds.

9. The method according to claim 7, wherein the nucleic acid isothermal amplification is conducted at 36-40° C. for 20-30 minutes.

10. The method according to claim 7, wherein the nucleic acid detection is conducted at 36-40° C. for 15-20 minutes.

11. The anti-pollution consumable according to claim 2, wherein there are one to three drain holes.

12. The anti-pollution consumable according to claim 2, wherein the outer reaction tube is made of polypropylene, and the sleeve and the inner reaction tube are made of polymethyl methacrylate (PMMA).

13. The anti-pollution consumable according to claim 2, wherein the inner reaction tube and the sleeve are integratedly synthesized.

14. The anti-pollution consumable according to claim 2, wherein the inner reaction tube and the sleeve are separately synthesized.
